(19) United States
(12) Patent Application Publication
(10) Pub. No.: US 2020/0110256 A1
(43) Pub. Date: Apr. 9, 2020

(54) OVERMOLDED DISTAL OPTICS FOR INTRALUMINAL OPTICAL PROBES

(71) Applicant: Canon U.S.A., Inc., Melville, NY (US)

(72) Inventors: Alexander Altshuler, Cambridge, MA (US); Tzu-Yu Wu, Malden, MA (US); Mitsuhiro Ikuta, Cambridge, MA (US)

(21) Appl. No.: 16/589,764

(22) Filed: Oct. 1, 2019

Related U.S. Application Data
(60) Provisional application No. 62/742,029, filed on Oct. 5, 2018.

(51) Int. Cl. G02B 23/24; A61B 1/00; A61B 1/06; A61B 1/07; G02B 23/02; G02B 23/26; G02B 27/10; A61B 5/00

(52) U.S. Cl. CPC G02B 23/2423; A61B 1/00009; A61B 1/00096; A61B 1/0661; A61B 1/07; A61B 5/0066; G02B 23/2453; G02B 23/2469; G02B 23/26; G02B 27/1086; G02B 23/02

(57) ABSTRACT

An optical probe includes: a tubular shaft having an opening extending from a proximal end to a distal end, a light guiding component arranged in the opening of the tubular shaft; and a distal optics component arranged distally to the light guiding component at the distal end of the tubular shaft. The distal optics component has a beam directing surface aligned with an optical axis of the light guiding component and at least one surface directly bonded to the distal end of the light guiding component and/or to the distal end of the tubular shaft. A light beam transmitted through the light guiding component is directed and shaped by the beam directing surface of the distal optics component. The distal optics component is directly molded over the distal end of the light guiding component and at least partially inside the tubular shaft.

### CROSS-REFERENCE TO RELATED APPLICATIONS

This application claims priority to U.S. Provisional Application Ser. No. 62/742,029 filed Oct. 5, 2018, the content of which is incorporated by reference in its entirety.

### BACKGROUND INFORMATION

### Field of the Disclosure

The present disclosure generally relates to medical devices. More particularly, the present disclosure relates to optical-fiber imaging probes, methods of manufacturing optical-fiber imaging probes, as well as systems and methods for imaging biological samples using such probes.

### Description of Related Art

Optical-fiber-based imaging probes, such as catheters and endoscopes, have been developed to access and image internal organs of humans and animals, and are now commonly used in various medical fields. Using an optical fiber based probe for imaging bodily lumens is getting more and more prevalent in a number of applications that can benefit from miniaturized probe sizes and high resolution images. In most of these applications, in order to provide a reasonable field of view, a rotating fiber with distal beam-forming optics is employed. Since these probes are required to be disposable in most medical applications, it is imperative to keep costs of such probes as low as possible, while maintaining high image quality.

Various methods have been previously disclosed for manufacturing miniature optical systems suitable for optical fiber imaging probes that provide some of the desired functionality described above. For example, in cardiology, optical coherence tomography (OCT), white light back-reflection, near infrared spectroscopy (NIRS) and fluorescence optical probes have been developed to obtain structural and/or molecular images of vessels and other bodily lumens with a catheter. An OCT catheter, which generally comprises a sheath, a torque coil and an optical probe inside the coil, is navigated through a lumen (e.g., a coronary artery), by manual or automatic control, to obtain intraluminal images. To miniaturize the size of the probe and improve image quality, for example, pre-grant publication US 2010/0253949 discloses an optical cap having a lensed surface configured to redirect light from a rotating probing fiber and focus the light outside of the cap. In another example, U.S. Pat. RE 45,512 discloses an OCT probe having a ball lens with multiple surfaces for reducing astigmatism of the focusing light.

Similarly, spectrally encoded endoscopy (SEE) is a technology that utilizes optical fibers, miniature optics, and a diffraction grating for high-speed imaging through small diameter and flexible endoscopic probes. Monochromatic or polychromatic light emanating from the diffraction grating at the distal end of an SEE probe is spectrally dispersed and projected in such a way that that each diffractive order or each color (wavelength) illuminates a different location of a sample (tissue) in one line (a spectrally-encoded dispersive line). Reflected light from the tissue can be collected and decoded by a spectrometer to form a line image, where each position of the line image corresponds to the specific wavelength of illumination. Spatial information in the other dimension perpendicular to the dispersive line is obtained by moving the probe using a motor, or by using a galvanometric scanner. SEE has been demonstrated to produce high quality images in two and three dimensions in monochromatic as well as in multiple color wavelengths. See for example U.S. Pat. No. 9,295,391.

Recent disclosures by the present applicant have addressed some aspects of certain needs for improvement in SEE imaging probes. For example, U.S. Pat. No. 10,288,868 discloses that, in an optical arrangement for forward viewing SEE, the imaging probe can comprise, a light guiding component, a light focusing component, a light reflecting component, and a simplified grating element along the probe optical axis, such that, when the light is transmitted through the grating component, at least one diffracted light propagates directly in a forward direction substantially parallel to the probe optical axis. In addition, U.S. Pat. No. 10,261,223 discloses a method for fabrication of a miniature endoscope using nanoimprint lithography. This patent discloses the fabrication method of an SEE endoscope by using nanoimprint lithography (micro-stamping) to form a diffractive patterned surface directly on the distal end of the probe. In one configuration, the optical probe includes an optical fiber and a gradient-index (GRIN) lens, and the method includes forming the diffractive configuration (a grating) directly on a distal end of the GRIN lens. Other related art for SEE imaging probes includes U.S. Pat. Nos. 8,145,018; 7,796,270; 7,859,679; 8,045,177; 8,812,087; 8,780,176.

However, these extremely small optical elements are fragile, difficult to handle, and prone to damage during manufacturing and operation. In particular, during operation, when the probe is immersed in fluids, such as water, contrast agents, blood, or stomach acid, or when the probe is rotated and/or translated at high speed in order to form an image, these extremely small optical elements can break or become detached. The overall effect of these drawbacks is that miniature optical systems are difficult to manufacture, prone to damage, and excessively expensive to be disposable. Therefore, there remains a need for fiber-optic-based imaging probes that can be fabricated easily, at low cost, and can maintain the ability to provide high quality images.

### SUMMARY OF EXEMPLARY EMBODIMENTS

According to at least one embodiment of the present disclosure a process of forming an optical probe includes overmolding a distal optics component over a light guiding component and/or a light focusing component. More specifically, the process includes (a) a step of inserting and positioning a distal end of a light guiding component such as an optical fiber inside a mold adapted to form a distal optics component having at least one beam directing surface; (b) a step of injecting molten optically transparent material (thermoplastic or glass) into the mold; (c) a step of allowing time for the injected material to solidify; and (d) an step opening the mold and removing the distal end of the light guiding component with the distal optics component directly molded on the distal end of the light guiding component.

According to at least one more embodiment, an optical probe includes a drive cable, a light guiding component arranged inside the driver cable, and a distal optics component formed directly over the end of the light guiding component by overmolding. The drive cable has a shape of a hollow shaft extending from a proximal end to a distal end of the probe. The light guiding component includes a single mode fiber or a multi-mode fiber, and the fiber can be a multi-clad fiber. The distal optics component is directly molded over the fiber end making the probe manufacturing less expensive in mass production. The molded distal optics component strengthens the connections at the interface between the light guiding component and other optical components of the probe (e.g., GRIN lens to fiber, and GRIN lens to spacer).

According to another embodiment, the optical probe further includes a mechanical housing bonded to the distal end of the drive cable. In this embodiment, the distal optics component includes, an optical spacer, a reflective surface, a focusing lens, and a lead-in end all formed in a single part molded at least partially inside the metallic housing and contacting directly the distal end of the optical fiber. The distal optics component may be injection molded out of transparent thermoplastic material or compression molded out of glass.

These and other objects, features, and advantages of the present disclosure will become apparent to persons of ordinary skill in the art upon reading the following detailed description of exemplary embodiments in conjunction with the enclosed drawings, and appended claims.

### BRIEF DESCRIPTION OF DRAWINGS

Further objects, features and advantages of the present disclosure will become apparent from the following detailed description when taken in conjunction with the accompanying figures showing illustrative embodiments of the present disclosure.

FIG. 1A is sectioned perspective view of an embodiment showing an exemplary SEE probe 100 having illumination optics and a molded distal optics component inside the mechanical housing and a drive cable. FIG. 1B is a perspective view illumination optics and a molded distal optics component of the SEE probe 100.

FIG. 2A, FIG. 2B and FIG. 2C show perspective views of an embodiment showing an exemplary SEE probe 100 having illumination optics and an overmolded distal optics component having angled surfaces.

FIG. 3A and FIG. 3B respectively show perspective and side views of illumination optics and an overmolded distal optics component having a curved reflective focusing surface.

FIG. 4 illustrates the light path through the illumination optics and overmolded distal optics component of a forward viewing SEE endoscope.

FIG. 5A is sectioned perspective view of an embodiment showing an exemplary SEE probe 100 having an overmolded distal optics component formed inside the mechanical housing separate from the illumination optics. FIG. 5B is sectioned perspective view of an embodiment showing an exemplary SEE probe 100 having an overmolded distal optics component formed partially inside the drive cable and separate from the illumination optics.

FIG. 6A, FIG. 6B, FIG. 6C, FIG. 6D are perspective views illustrating a process of adding a diffractive component to the distal end of an overmolded distal optics component.

FIG. 7 illustrates a partially-cutaway view of an example embodiment of an OCT imaging probe 700 that includes overmolded distal optics component in a mechanical housing.

FIG. 8 illustrates a partially-cutaway view of an example embodiment of an OCT probe 800 that includes overmolded distal optics component formed partially inside the drive cable.

FIG. 9 illustrates a partially-cutaway view of an exemplary embodiment of an OCT probe 900 that includes overmolded distal optics component formed partially inside a tubular mechanical housing and over a distal end of the light guiding component.

FIG. 10A illustrates an example embodiment of an OCT system using an OCT imaging probe. FIG. 10B shows a functional diagram of computer applicable to control and operate the OCT system. FIG. 10C illustrates an example embodiment of an SEE system using an SEE probe.

### DETAILED DESCRIPTION OF EXEMPLARY EMBODIMENTS

The exemplary embodiments disclosed herein are based on an objective of providing micron-sized fiber-optic-based imaging probes that can be fabricated easily, at low cost, and can maintain the ability to provide high quality images. As used herein, micron-sized imaging probes and optical elements thereof may refer to components having physical dimensions 1.5 millimeter (mm) or less in diameter.

Throughout the figures, the same reference numerals and characters, unless otherwise stated, are used to denote like features, elements, components or portions of the illustrated embodiments. In addition, while the subject disclosure is described in detail with reference to the enclosed figures, it is done so in connection with illustrative exemplary embodiments. It is intended that changes and modifications can be made to the described exemplary embodiments without departing from the true scope and spirit of the subject disclosure as defined by the appended claims. Although the drawings represent some possible configurations and approaches, the drawings are not necessarily to scale and certain features may be exaggerated, removed, or partially sectioned to better illustrate and explain certain aspects of the present disclosure. The descriptions set forth herein are not intended to be exhaustive or otherwise limit or restrict the claims to the precise forms and configurations shown in the drawings and disclosed in the following detailed description.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached”, “coupled” or the like to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown in one embodiment can apply to other embodiments. It will also be appreciated by those of skill in the art that references to a structure or feature that is disposed “adjacent” to another feature may have portions that overlap or underlie the adjacent feature.

The terms first, second, third, etc. may be used herein to describe various elements, components, regions, parts and/or sections. It should be understood that these elements, components, regions, parts and/or sections are not limited by these terms of designation. These terms of designation have been used only to distinguish one element, component, region, part, or section from another region, part, or section. Thus, a first element, component, region, part, or section discussed below could be termed a second element, component, region, part, or section merely for purposes of distinction but without limitation and without departing from structural or functional meaning.

As used herein, the singular forms “a”, “an”, and “the” are intended to include the plural forms as well, unless the context clearly indicates otherwise. It should be further understood that the terms “includes” and/or “including”, “comprises” and/or “comprising”, “consists” and/or “consisting” when used in the present specification and claims, specify the presence of stated features, integers, steps, operations, elements, and/or components, but do not preclude the presence or addition of one or more other features, integers, steps, operations, elements, components, and/or groups thereof not explicitly stated. Further, in the present disclosure, the transitional phrase “consisting of” excludes any element, step, or component not specified in the claim. It is further noted that some claims or some features of a claim may be drafted to exclude any optional element; such claims may use exclusive terminology as “solely,” “only” and the like in connection with the recitation of claim elements, or it may use of a “negative” limitation.

The term “about” or “approximately” as used herein means, for example, within 10%, within 5%, or less. In some embodiments, the term “about” may mean within measurement error. In this regard, where described or claimed, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range, if recited herein, is intended to include all sub-ranges subsumed therein. As used herein, the term “substantially” is meant to allow for deviations from the descriptor that do not negatively affect the intended purpose. For example, deviations that are from limitations in measurements, differences within manufacture tolerance, or variations of less than 5% can be considered within the scope of substantially the same. The specified descriptor can be an absolute value (e.g. substantially spherical, substantially perpendicular, substantially concentric, etc.) or a relative term (e.g. substantially similar, substantially the same, etc.).

The present disclosure generally relates to medical devices, and it exemplifies embodiments of an optical probe which may be applicable to a spectroscopic apparatus (e.g., an endoscope), an optical coherence tomographic (OCT) apparatus, or a combination of such apparatuses (e.g., a multi-modality optical probe). The embodiments of the optical probe and portions thereof are described in terms of their state in a three-dimensional space. As used herein, the term “position” refers to the location of an object or a portion of an object in a three-dimensional space (e.g., three degrees of translational freedom along Cartesian X, Y, Z coordinates); the term “orientation” refers to the rotational placement of an object or a portion of an object (three degrees of rotational freedom—e.g., roll, pitch, and yaw); the term “posture” refers to the position of an object or a portion of an object in at least one degree of translational freedom and to the orientation of that object or portion of object in at least one degree of rotational freedom (up to six total degrees of freedom); the term “shape” refers to a set of posture, positions, and/or orientations measured along the elongated body of the object. As it is known in the field of medical devices, the terms “proximal” and “distal” are used with reference to the manipulation of an end of an instrument extending from the user to a surgical or diagnostic site. In this regard, the term “proximal” refers to the portion of the instrument closer to the user, and the term “distal” refers to the portion of the instrument further away from the user and closer to a surgical or diagnostic site.

As used herein the term “catheter” generally refers to a flexible and thin tubular instrument made of medical grade material designed to be inserted through a narrow opening into a bodily lumen (e.g., a vessel) to perform a broad range of medical functions. The more specific term “optical catheter” refers to a medical instrument comprising an elongated bundle of one or more flexible light conducting fibers disposed inside a protective sheath made of medical grade material and having an optical imaging function. A particular example of an optical catheter is fiber optic catheter which comprises a sheath, a coil, a protector and an optical probe. In some applications a catheter may include a “guide catheter” which functions similarly to a sheath.

As used herein the term “endoscope” refers to a rigid or flexible medical instrument which uses light guided by an optical probe to look inside a body cavity or organ. A medical procedure, in which an endoscope is inserted through a natural opening, is called an endoscopy. Specialized endoscopes are generally named for how or where the endoscope is intended to be used, such as the bronchoscope (mouth), sigmoidoscope (rectum), cystoscope (bladder), nephroscope (kidney), bronchoscope (bronchi), laryngoscope (larynx), otoscope (ear), arthroscope (joint), laparoscope (abdomen), and gastrointestinal endoscopes.

In the present disclosure, the terms “optical fiber”, “fiber optic”, or simply “fiber” refers to an elongated, flexible, light conducting conduit capable of conducting light from one end to another end due to the effect known as total internal reflection. The terms “light guiding component” or “waveguide” may also refer to, or may have the functionality of, an optical fiber. The term “fiber” may refer to one or more light conducting fibers. An optical fiber has a generally transparent, homogenous core, through which the light is guided, and the core is surrounded by a homogenous cladding. The refraction index of the core is larger than the refraction index of the cladding. Depending on design choice some fibers can have multiple claddings surrounding the core.

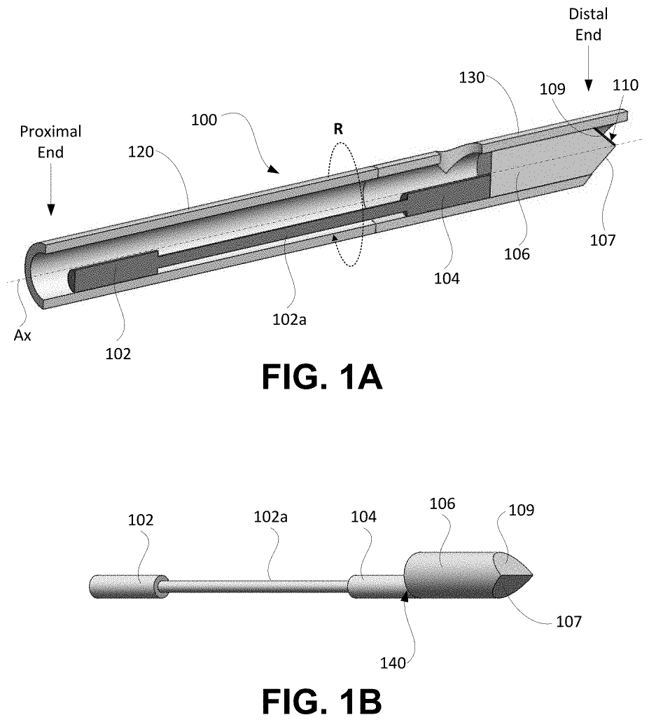

Turning now to the specific embodiments shown in the drawings, FIG. 1A and FIG. 1B show a first embodiment of an optical imaging probe. FIG. 1A particularly illustrates illumination optics arranged in a distal portion of an exemplary SEE probe 100. The SEE probe 100 has a proximal end and a distal end, and is comprised of at least a light guiding component 102 (e.g., single mode fiber, multi-mode fiber, where the fiber is a single or double clad fiber), a focusing component 104 (e.g., a GRIN lens, ball lens, a reflective curved surface, etc.), a distal optics component (e.g., an spacer 106) which includes a dispersive or diffractive component 110 (e.g., a grating, prism, or the like). In this embodiment, the light guiding component 102, the focusing component 104, and distal optics component (spacer 106) are all enclosed inside a drive cable 120 and a mechanical housing 130. The drive cable 120 has a shape of a hollow or tubular shaft with a circular opening extending from the proximal end to the distal end of the probe. The mechanical housing 130 is a cylindrical tube (e.g., a metallic can) fixedly attached to the distal end of the drive cable 120 such that both the mechanical housing 130 and drive cable 120 share the same (common) longitudinal axis (Ax). The first light guiding component 102 may be an optical fiber having a stripped portion 102a. The mechanical housing 130 is attached (e.g., welded or glued or otherwise bonded) to the distal end of the drive cable 120. In some embodiments, the mechanical housing or can may be obviated,

The first light guiding component 102 is spliced or glued to the focusing component 104; and the focusing component 104 is in turn bonded or glued to the spacer 106 (e.g., a coreless fiber, a transparent optical grade glass or plastic). The spacer 106 has a substantially cylindrical shape, and at least part of its cylindrical surface is fixedly attached to the inner surface of the mechanical housing 130. The focusing component 104 is aligned off-center with respect to the optical axis (Ox) of spacer 106. The spacer 106 has two surfaces (a first surface 107 and a second surface 109) at the distal end thereof. The first surface 107 and second surface 109 may be polished at specific angles to direct the light from the focusing component 104 in a predetermined direction. However, as explained later in more detail, the spacer 106 along with its first and second surfaces 106 and 107 may be directly molded as a single element part inside the mechanical housing 130. The first surface 107 is or includes a reflective surface (e.g., a mirror coating or total-internal-reflection surface) which reflects the light from the focusing component 104 towards the second surface 109. A grating structure or the like is provided as the diffractive component 110 on the second surface 109. In some embodiments, the diffractive component 110 is fabricated by micro-stamping a suitable material to form a diffractive grating, and the grating disperses the light to a predetermined field of view. The mechanical housing 130 is provided at the distal end of the drive cable 120 in the form of a metallic can for protection and positioning of the distal optics. The proximal end of the mechanical housing 130 is rigidly attached to a flexible coil of the drive cable 120. The drive cable 120 is used for transmitting rotational torque from a non-shown motor to the first light guiding component 102 (optical fiber) and/or to the distal optics (focusing component 104 and spacer 106). The metallic can or mechanical housing 130 is preferably welded or soldered or otherwise attached to the distal end of drive cable 120.

As shown in FIG. 1B, the focusing component 104 is joined to the spacer 106 in an off-centered manner by mechanically joining the two components at a joint 140 by splicing and/or gluing techniques. Splicing is a well known technique of joining two fiber optic cables together by either fusion splicing or mechanical splicing. Mechanical splicing uses alignment devices designed to hold the ends of the two fiber components in a precisely aligned position thus enabling light to pass from one fiber end into the other component with a typical loss of about 0.3 dB. Mechanical splicing does not create a permanent bond, so it is not applicable to joining the focusing component 104 to the spacer 106. In fusion splicing a machine is used to precisely align the two fiber components so that the glass ends are “fused” or “welded” together using some type of heat or electric arc. This produces a continuous (permanent) connection between the two ends of the fiber components enabling very low loss of light transmission (e.g., a transmission typical loss in fusion splicing of two fiber ends is about 0.1 dB). In the field of OCT probes, the same type of fusion splicing used in conventional fiber optics has been applied to attach and align optical components. See for example, U.S. Pat. No. 6,445,939 and pre-grant publication US 2006/0067620. However, fusion splicing and/or bonding of fiber components may sometimes exhibit mechanical failures and/or optical transmission losses of up to 0.5 dB.

To obviate such drawbacks of splicing and aligning, the spacer 106 illustrated in FIG. 1A and FIG. 1B can be directly molded inside the mechanical housing 130 abutting directly against the distal end of the focusing component 104. The spacer 106 can be injection molded out of transparent thermoplastic material or compression molded out of glass. In this manner, spacer 106 having the first surface 107 and second surface 109 (a distal optics component) is integrally formed directly on the distal end of the focusing component 104, such that at least one surface of the spacer 106 is directly formed on the end surface of the focusing component. This process at least provides a more robust joint 140 and improves alignment between the distal optics component and the fiber and lens assembly.

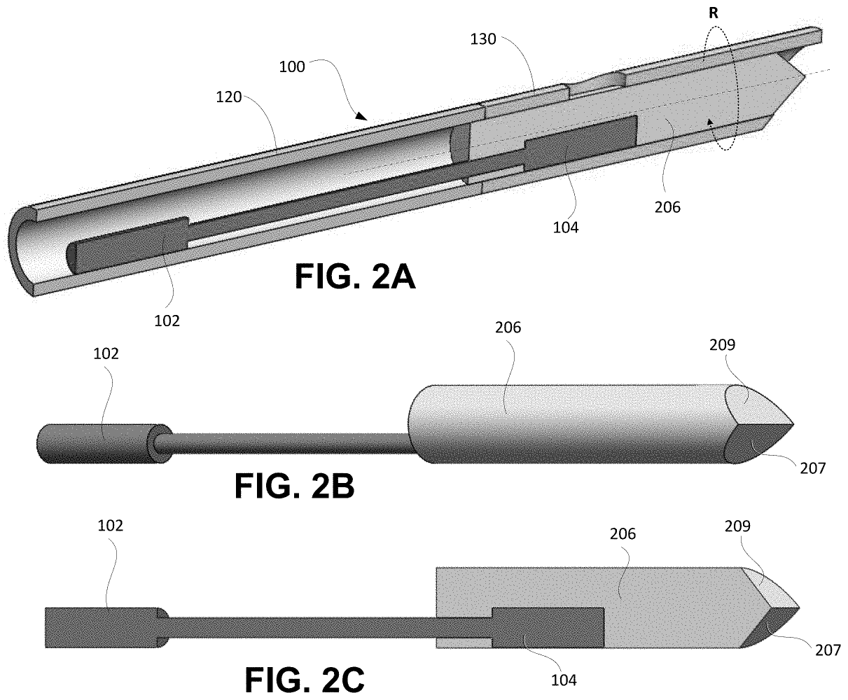

FIG. 2A, FIG. 2B, and FIG. 2C shows various views of another embodiment of the optical probe 100 where a spacer 206 is integrally formed around the focusing component 104 and bonded to the light guiding component 102 to obviate the drawbacks of splicing and alignment techniques. Specifically, according to the embodiment shown in FIGS. 2A, 2B, and 2C, the spacer 206 is molded over the focusing component 104 (e.g., a GRIN lens) to more efficiently create a fiber-distal optics assembly. In this embodiment, the spacer 206 with two angled surfaces (a first angled surface 207 and a second angled surface 209) is directly molded over the GRIN lens and a fiber-distal optics assembly of a more robust structure is obtained. In some embodiments, the part of the spacer 206 may be injection molded out of transparent thermoplastic material or compression molded out of glass. This process not only secures the joining of the first light guiding component 102 to the focusing component 104, and improves the joining of the focusing component 104 to the spacer 206, but it also reduces the fabrication steps for forming the first and second angled surfaces. More specifically, since the spacer 206 is molded over the already joined fiber and GRIN lens, the process can obviate the polishing of the two angle surfaces. That is, because the spacer 206 is molded over the focusing component 104 and at least part of the first light guiding component 102, the spacer 206 also strengthens the connection joints between the fiber end to GRIN lens, and between the GRIN lens to the spacer. This process also avoids the step of attaching the GRIN lens assembly to the spacer by epoxy. Moreover, by overmolding the spacer 206 over the fiber and GRIN lens, a process of alignment and polishing of the joining surfaces is improved.

The grating structure of the diffractive component 110 can be either molded together with the spacer 206, e.g., with a microstructure insert, or it can be stamped onto the second angled surface 109 after the molded part is formed. For example, the method of forming a diffractive configuration as disclosed in U.S. Pat. No. 10,261,223, which is incorporated by reference herein, can be used for forming the diffractive component 110 using nanoimprint lithography.

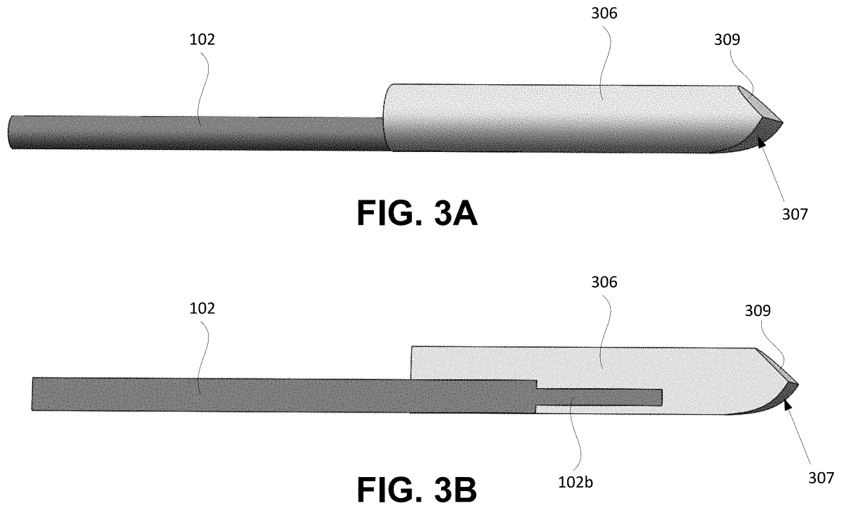

FIGS. 3A and 3B illustrate a further embodiment where both a focusing component 304 and a spacer 306 are molded over the first light guiding component 102. In this embodiment, the optical spacer 306 includes a first surface 307 for focusing optics and a second surface 309 for dispersive optics all formed in a single piece molded over the light guiding component 102 which is comprised of an optical fiber with a stripped portion 102b. To provide a focusing effect, the focusing optics is molded with a curvature to provide a positive optical power to collimate and slightly focus the diverging light emitted from the first light guiding component 102 (e.g., single mode fiber). The light emitted through the distal end of the first light guiding component 102 may be reflected by total internal reflection (TIR) or by a reflective coating on the curvature of the first surface 307. On the second surface 309, the diffractive component (e.g., a grating) can be either molded together with the spacer 306 or it be fabricated (e.g., using micro-stamping) after the spacer 306 is molded over the fiber. In this embodiment, therefore, the focus component is not provided in direct contact with the distal end of first light guiding component 102.

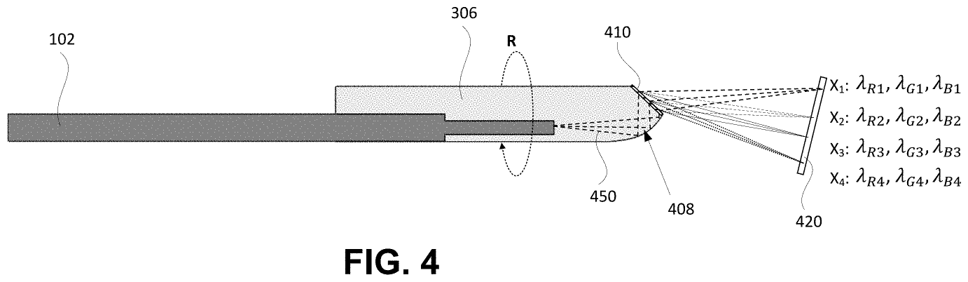

FIG. 4 illustrates the light path of the illumination portion of an exemplary forward-viewing SEE probe. As shown in FIG. 4, a light beam 450 is emitted through the distal end of the light guiding component 102, is expanded within the spacer 306, and is reflected and focused by a curved reflective surface 408 towards the upper angled surface. The light is incident on a diffractive component 410 (a grating element) formed on the second surface. The diffractive component 410 causes the light beam 450 to be dispersed into an illumination line 420 where different wavelengths of light are focused at different spatial locations. In a color imaging application, 3 bands of light spectrum (λ_{R}, λ_{G}, λ_{B}) with different diffraction orders are overlapped and focused on the illumination line 420. An example of this process is described, for example, in “Single-beam spectrally encoded color imaging,” by Mitsuhiro Ikuta et al., Opt. Lett. 43, 2229-2232 (2018).

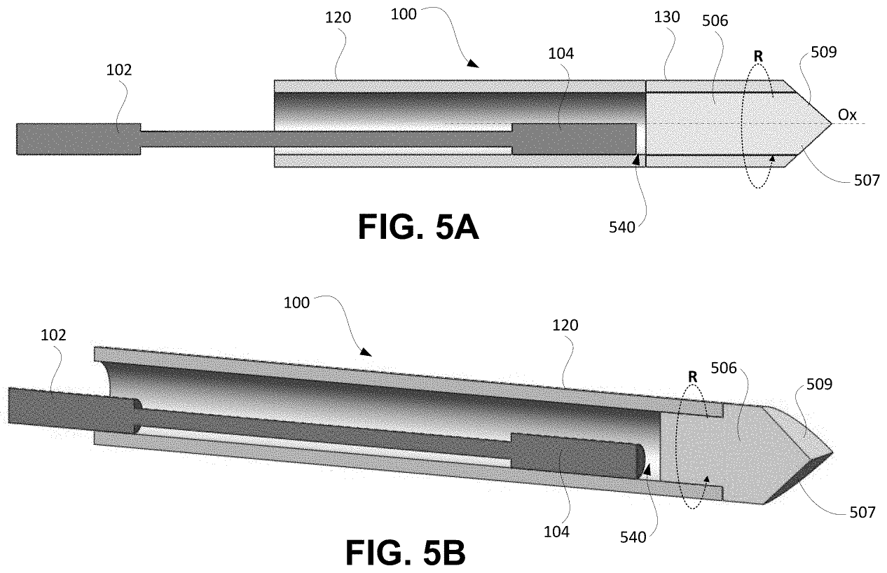

FIG. 5A and FIG. 5B show a further embodiment of molded SEE distal optics formed directly at the distal end of the drive cable 120, but not in contact with the light guiding component 102. In this embodiment, the optical spacer 506 includes a first surface 507 and a second surface 509. The embodiment shown in FIG. 5A is similar to the embodiment shown in FIG. 1A in that the optical spacer is directly molded inside the mechanical housing 130 (a metallic can), except that the optical spacer 506 is formed at a predetermined distance 540 separated from the fiber and GRIN lens assembly. In this case too, the grating can be molded by microstructure insert or stamped afterward on the second surface (upper angled surface) of the spacer 506.

In the embodiment shown in FIG. 5A, the optical spacer 506 is molded inside the metal can (mechanical housing 130), which is attached to the distal end of drive cable 120. The optical spacer 506 is rotated by the torque transmitted from the drive cable 120, while the light guiding component 102 and the focusing component 104 (fiber and GRIN lens assembly) remain stationary. An example of this type of endoscopic probe having a fixed fiber and rotating distal optics is disclosed in U.S. Pat. No. 10,321,810 to Ikuta et al., which is incorporated by reference herein for all purposes. Although FIG. 5A shows a configuration where the optical axis of optical fiber-GRIN lens assembly is off-centered from the rotational axis of the probe, the molded optical spacer 506 can be made of a different size such that the optical axis of the GRIN lens assembly is aligned with the mechanical axis of the probe. Alternatively, in the case where the GRIN lens illuminates the optical spacer at an off-center position, the mechanical housing 130 and/or the drive cable 120 can include a mechanical spacer with an off-centered hole so that the light from GRIN lens illuminates at the off-centered position of the can. In the case of a large spacer and a centered GRIN lens, either the GRIN lens or the inside of can, or the inside of the drive cable, may have a mechanical collar (fiber centering feature) for aligning the focus component 104 such that the GRIN lens stays centered in the hollow shaft of the probe.

FIG. 5B shows another example of the embodiment where the optical spacer 506 is directly molded inside the distal end of drive cable 120. Specifically, as shown in FIG. 5B, the optical spacer 506 is molded at least partially inside the inner diameter of the drive cable 120 at the distal end thereof. In this manner, the optical spacer 506 can be rotated with the drive cable 120 while the light guiding component 102 and the focusing component (GRIN lens) 104 can remain stationary. Again to maintain alignment of the non-rotating elements (light guiding component 102 and focusing component 104), a mechanical collar can be provided inside the inner diameter of the drive cable 120. Either of the examples shown in FIG. 5A or FIG. 5B can be made similar to the embodiment of FIG. 4 where the reflecting surface of the overmolded optical spacer 506 may have positive optical power, and the upper angled surface may have a diffractive component. An advantage of this configuration where the optical spacer 506 is directly molded within the distal end of the probe (inside the can or the drive cable) and the light guiding component is maintained stationary is to eliminate the need for a fiber optic rotary junction because the fiber is not continuously rotating at the distal end.

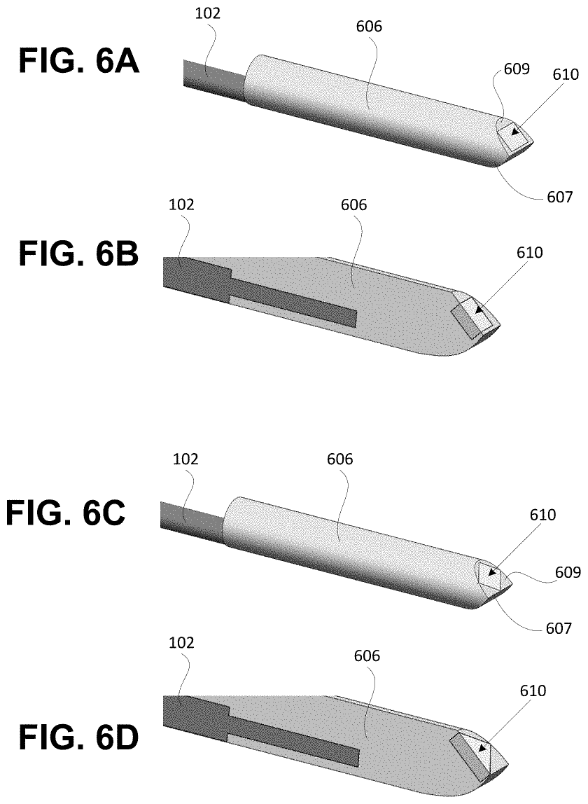

FIGS. 6A, 6B, 6C, and 6D illustrate various examples of a diffractive component (e.g., a grating) which is a separately made overmolded component. FIGS. 6A and 6B respectively show a perspective and cross-sectional view of a spacer 606 overmolded over a light guiding component 102. The overmolded optical spacer 606 has at the distal end thereof a first surface 607 and a second surface 609. Here, to form a diffractive component 610 a rectangular-shaped grating chip is provided on the second surface 609. Similarly, FIGS. 6C and 6D respectively show a perspective and cross-sectional view of a spacer 606 overmolded over a light guiding component 102. The overmolded optical spacer 606 has at the distal end thereof a first surface 607 and a second surface 609. Here, to form a diffractive component 610 a rhombus-shaped grating chip is provided on the second surface 609.

Therefore, according to at least one embodiment, a grating component can be etched (or produced by any other method of mass-production) on a thin glass wafer, then diced into individual chips, and an individual chip can be inserted into the mold to be overmolded as one piece with a spacer. This process could allow for a more robust assembly and, at the same time, lower manufacturing cost. Alternatively, the grating component can be added as a final step in a molding process of forming the optical spacer 606. Specifically, the above described individually diced grating chip can be added to the material of the optical spacer 606, as a final step of the molding process, but before the spacer material is solidified. Adding the grating component 610 to the optical spacer 606, during or after the distal optics assembly is molded, as shown in FIG. 6A-6D, may be implemented with any of the above-described embodiments.

As noted in the background section, the present disclosure acknowledges that extremely small optical elements are fragile, difficult to handle, and prone to damage during manufacturing and operation. There remains a need for fiber-optic-based imaging probes that can be fabricated easily and at low cost while maintaining the ability to provide high quality images. However, certain miniature optical systems are difficult to manufacture, are prone to damage, and are excessively expensive to be disposable. The foregoing embodiments address at least some of those needs, by disclosing a novel distal optics assembly (e.g., a spacer and a prism or grating, and/or focusing optics, and/or dispersive optics) formed directly over the end of a fiber optics assembly (e.g., single mode fiber, GRIN lens attached to a fiber, a GRIN lens attached to a spacer then to a fiber as fiber optics assembly, etc.) by overmolding a single distal optics component at the distal end of drive cable component or the mechanical housing component.

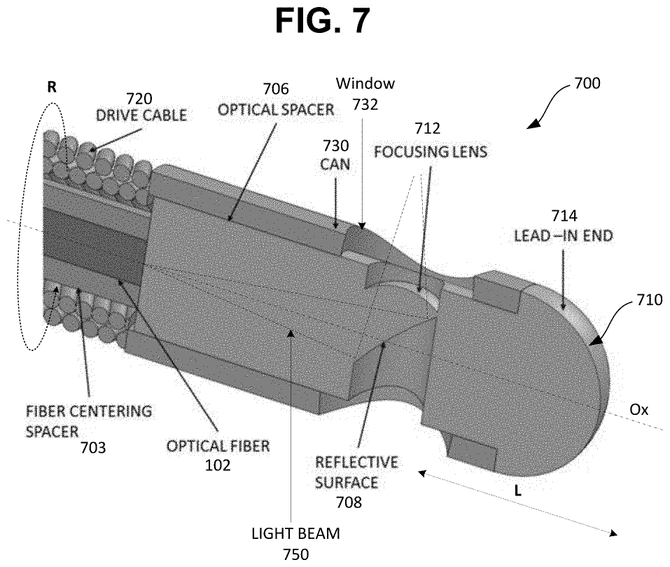

FIG. 7 illustrates an exemplary embodiment of distal optics in an OCT imaging probe 700. The OCT probe 700 includes a light guiding component 102 (an optical fiber) arranged inside a drive cable 720, and a molded component 710 arranged inside a mechanical housing (or can) 730. The light guiding component 102 is an optical fiber contained within the drive cable 720 and surrounded by a fiber centering spacer 703. The single molded component 710 includes an optical spacer 706, a reflective surface 708, a focusing lens 712, and a lead-in feature or cap 714 all formed integrally as the single molded component 710 and arranged inside the mechanical housing 730 (metallic can). In this embodiment. In this embodiment, the proximal end of optical spacer 706 is arranged in direct contact with the distal end of the light guiding component 102 (optical fiber). This single molded component 10 may be injection molded out of transparent thermoplastic material, or it may be compression molded out of optical grade glass material. A light beam 750 emitted through the distal end of light guiding component 102 is expanded within the spacer 706, is reflected by the reflective surface 708, and is focused by the focusing lens 712 at a working distance away from the optical axis Ox. In this manner, light passing through the light guiding component 102 is incident on the reflective surface 708 (beam directing surface), and the light is directed in a direction angular to the optical axis.

The OCT probe 700 is configured to rotate or oscillate in a direction of arrow R around its probe optical axis Ox, and to linearly translate along a direction L substantially parallel to the probe optical axis Ox. To that end, the drive cable 720 and the optical-focusing component are fixed relative to each other. The drive cable 720 delivers rotational torque from a non-shown torque drive unit at its proximal end to its distal end in order to spin the distal end of the probe, which is attached to the optical-focusing component to create an OCT 3D scan.

At the distal end of the optical-imaging probe 700 is the lead-in feature or cap 714. This lead-in feature or cap 714 is preferably atraumatic and configured to provide a guiding surface for safe advancement of the optical probe through lumens such as blood vessels and the like. Thus, a half-ball shaped lead-in end may be positioned at the distal end of the mechanical housing 730 to facilitate safe probe advancement. The lead-in feature or cap 714 may be, for example, a rounded tip integrally formed (molded) with the rest of the optical-focusing component. The lead-in feature or cap 714 has a soft rounded-off profile to minimize trauma to a blood vessel wall.

Also provided in this particular embodiment is the fiber centering spacer 703 located concentrically between the light guiding component 102 and the drive cable 720. The centering spacer 703 may be used to ensure precise position and alignment of the optical axis relative to the axis of rotation of the fiber.

The optical-focusing component is located inside a mechanical housing or can 730 for protection and positioning. The can 730 is a cylindrical tube surrounding the optical-focusing component. The proximal end of the can is rigidly attached to a flexible wound drive cable 720 transmitting rotational motion from a non-illustrated torque source to the optical fiber and the distal optics. In some embodiments, the can 730 is preferably welded or soldered to the drive cable 720. In some embodiments, at least one surface of the optical-focusing component is directly formed on at least one surface of the can 730. The mechanical housing 730 includes an opening or window 732 through which the light beam 750 is focused outside of the sheath (not shown).

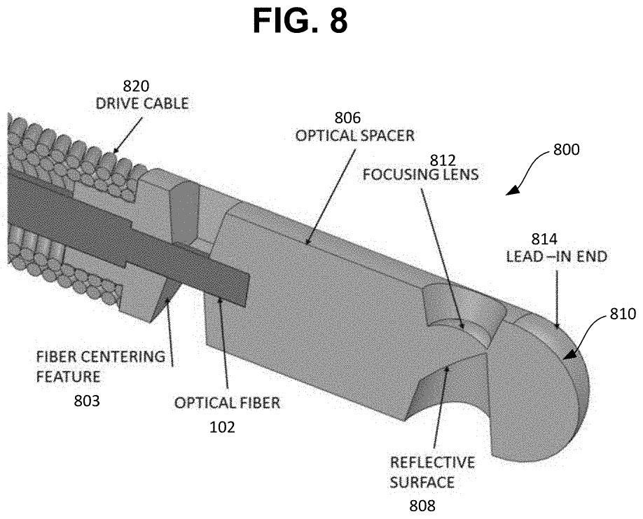

FIG. 8 illustrates a further embodiment of distal optics in an OCT imaging probe 800. In FIG. 8, the probe 800 includes a light guiding component 102 enclosed inside a flexible coil-based drive cable 820, and a molded distal optics component 810 which is at least partially inserted within the inner diameter of the distal end of drive cable 820. In this embodiment, the mechanical housing (a metallic can) is not provided at the distal end of the drive cable 820. In addition, at least part of a fiber centering feature 803 is incorporated into the molded light-focusing component 810. More specifically, in the embodiment of FIG. 8, the molded light-focusing component 810 includes a centering feature 803 (centering portion), an optical spacer 806, a reflective surface 808, a focusing lens 812, and a lead-in end or cap 814, which are all molded together as a single component and attached to the distal end of the drive cable 820. By forcing part of the molding material directly into at least a portion of the inner surface of the drive cable 820 a strong bond is created between the distal end of drive cable 820 and the light focusing-component 810 to assure mechanical integrity of the probe as a whole. To maintain alignment accuracy of the optical spacer 806, reflective surface 808, focusing lens 812, and lead-in feature 814, an additional mold feature (a guide) may be used to center the optical fiber inside the mold prior to material injection.

The forming of the molded light-focusing component is not limited to the embodiments shown in FIG. 7 and FIG. 8. In another embodiment an additional glass spacer may be spliced to the end of optical fiber prior to overmolding to prevent molding material overheating the fiber exit point. In yet another embodiment a ball lens may be formed at the end of the optical fiber prior to overmolding the distal optical component to prevent molding material overheating the fiber exit point and to minimize back reflections.

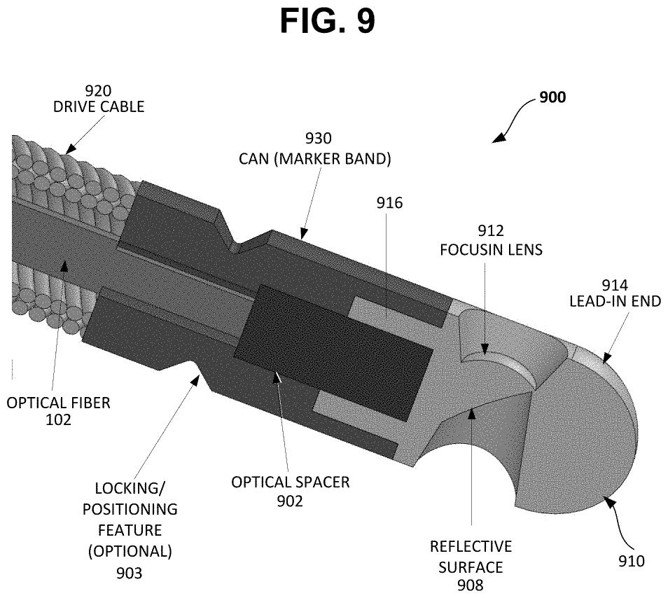

FIG. 9 illustrates an exemplary embodiment of an OCT imaging probe 900 comprising an overmolded distal optics assembly according to present disclosure. The OCT probe 900 includes a flexible multi-layer coil drive cable 920, a light guiding component 102 arranged inside the drive cable 920, and a molded distal optics component 910, which is arranged at least partially inside a tubular mechanical housing (or can) 930. The light guiding component 102 is an optical fiber contained within the drive cable 920 and extending into the tubular mechanical housing (or can) 930. The light guiding component 102 is comprised of an optical fiber and an optical spacer 902 which is directly and coaxially affixed to the distal end of the optical fiber. The distal optics component 910 includes a connecting portion 916 formed to fixedly surround the optical spacer 902, a reflective surface 908 (beam directing surface), a focusing lens 912, and a lead-in feature (or atraumatic cap) 914 all of which are formed integrally as single molded component. In this embodiment, the distal end of the light guiding component 102 is arranged in direct contact with (abutting against) the optical spacer 902. For example, the optical spacer 902 is a glass cylinder mechanically-spliced or fusion-spliced to the distal end of the optical fiber. The single piece distal optics component 910 may be injection molded out of transparent thermoplastic material, or it may be compression molded out of optical grade glass material. Examples of thermoplastic material applicable to the embodiments disclosed herein include optical grade polymethyl-methacrylate (PMMA) and polycarbonate (PC), and an example of glass material includes borosilicate.

Also provided in this particular embodiment is an optional locking or positioning feature 903 which is a “V” shaped notch formed around the outer surface of the tubular mechanical housing or can 930. The “V” shaped notch or groove like feature on the outside of the probe can allow to precisely position and positively lock the can in the mold. The entire tubular mechanical housing (or can) 930, or at least a portion thereof, can be made of radiopaque material such as Platinum or Iridium. Therefore, the tubular mechanical housing 930 may function as a marker band used to identify and/or track the location of the optical probe during imaging procedures.

In yet another embodiment the end of the optical fiber may be cleaved and/or polished at an angle other than normal to the optical axis prior to overmolding to minimize back reflections. In yet another embodiment the end of the optical fiber may be coated with an antireflection coating prior to overmolding to minimize back reflections. The distal optics component is directly overmolded over the fiber end making its position more precise and probe manufacturing less expensive in mass production.

In the various embodiments described above, the distal optics component molded as a single component can be disposable. The distal optics component may be made compression molded glass or injection molded optical-grade plastics, which can be manufactured relatively inexpensively such that the distal optics component may be disposed of after a single use and remain cost-effective in comparison with conventional optical probe designs. In certain embodiments, the distal optics component may be sterilizable.

As noted in the background section, the present disclosure acknowledges that extremely small optical elements are fragile, difficult to manufacture and handle, and prone to damage during manufacturing and operation. Also, conventional optical imaging probes tend to be excessively expensive to be disposable. The foregoing embodiments address at least some of these issues, by disclosing a novel distal optics assembly (e.g., a spacer and a prism or grating, and/or focusing optics, and/or dispersive optics) formed directly over the end of a fiber optics assembly (e.g., single mode fiber, GRIN lens attached to a fiber, a GRIN lens attached to a spacer then to a fiber as fiber optics assembly, etc.) by overmolding a single distal optics component at the distal end of a drive cable component.

The overmolded distal optics component comprises a spacer and at least one dispersive component. The overmolded distal optics component comprises a spacer and at least one beam directing surface. The beam directing surface is a TIR surface or a surface coated with reflective coating. The beam directing surface has a dispersive component such as a microstructural diffractive grating on it.

In some embodiments, the distal optics component is molded directly over the end of the fiber optics assembly so as to be disposed, at least partially, inside a mechanical housing attached to the drive cable. The distal optics comprises at least one beam directing surface which has positive optical power.

In at least one embodiment, the distal optics component is formed on the drive cable separated from the fiber optics assembly. The distal optics comprises at least one dispersive component. The dispersive component includes a grating which is made separately from the overmolded component and is added to the distal optics component assembly after the molding process.

The process of overmolding the distal optics component over the light guiding component and/or the light focusing component may include (a) a step inserting and positioning a distal end of the light guiding component inside a mold adapted to form a distal optics component having at least one beam directing surface; (b) injecting molten optically transparent material (thermoplastic or glass) into the mold; (c) allowing time for the injected material to solidify; and (d) opening the mold and removing the distal end of the light guiding component with the distal optics component directly molded on the distal end of the light guiding component. The foregoing process will result in the formation of an imaging core as that illustrated in FIGS. 1B, 2B, 2C, 3A, and 3B. The imaging core can then be assembled inside the drive cable with or without the protective mechanical can. However, in other embodiments, distal optics component can be molded directly inside the tubular shaft of the guide wire and/or the mechanical housing, as shown in FIGS. 5A, 5B, and 7-9.

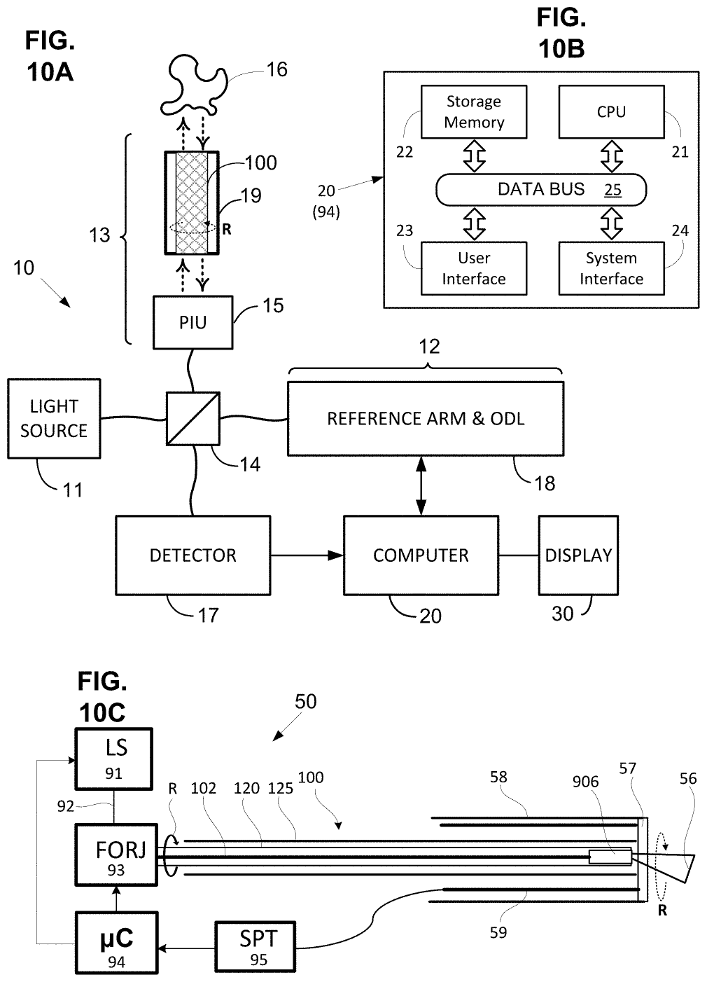

FIG. 10A illustrates an embodiment of an exemplary imaging system configured to use an optical probe for intraluminal imaging. The imaging system 10 shown in FIG. 10A is an interferometric OCT system which includes a light source 11, a reference arm 12, a sample arm 13, a beam splitter 14, and one or more detectors 17. The light source 11 emits light, and the light source 11 may be, for example, a broad-band light source with a short coherence length, a superluminescent light-emitting diode (SLED), a tunable light source, a supercontinuum light source, and a white-light source. The beam splitter 14 splits the light, directs part of the light to the reference arm 12, and directs part of the light to the sample arm 13. In some embodiments, the OCT system 10 may use one or more circulators to split the light and use one or more beam couplers to recombine the light. The beam splitter 14 can be a fiber-optic beam splitter with a split ratio of 50:50 (±designer tolerance) or any other ratio as appropriate.

The sample arm 13 includes a patient-interface unit 15 and an optical-imaging device 19. The optical-imaging device 19 includes an optical probe 100, which directs a beam of light to a sample 16 and detects light that is reflected from or scattered by the sample 16. The optical probe 100 then transmits the reflected or scattered light back to the beam splitter 14.

The reference arm 12 can include conventional optics and an optical delay line 18. The optical delay line 18 includes a mirror, and light that travels through the optical delay line 18 is reflected off the mirror and travels back to the beam splitter 14. The sample and reference arms in the interferometer could consist of free-space optics, photonic integrated circuits, fiber-optics or combinations thereof, and the interferometer could have different architectures such as Michelson, Mach-Zehnder, or common-path interferometer designs.

A sample beam from the sample arm 13 and a reference beam from the reference arm 12 are recombined by the beam splitter 14, which generates a recombined beam that has an interference pattern (an interference pattern occurs when the reference arm and the sample arm have the same optical length). The recombined beam is detected by the one or more detectors 17 (e.g., photodiodes, photomultiplier tubes, a linear CCD array, an image sensor, a CCD array, a CMOS array) which convert the intensity of the interference pattern in an electrical signal. A computer 20 receives and processes the signal from the detector 17, and a display 30 provides a user with a resulting images and/or data obtained by the OCT system 10.

More specifically, the OCT system 10 is computer controlled by the computer 20 which includes one or more processors (e.g., one or more than one central processing unit or CPU) and associated circuitry to provide signaling commands for timing and control, and to process the interferometric data received from detector 17 into images or volumetric data. Specifically, the electrical signals from the detector 17 are transferred to the computer 20 for processing and display. The computer 20 may contain, in addition to a CPU, for example, one or more of a field-programmable gate array (FPGA), a digital signal processor (DSP), an application specific integrated circuit (ASIC), a graphic processing unit (GPU), a system on chip (SoC) or a combination thereof, which performs some or the entire image processing and signaling of the OCT system 10.

FIG. 10B shows a schematic block diagram of a computer 20 applicable to the various operating aspects of the OCT system 10. To that end, the computer 20 includes or is operably attached to a display 30 for displaying images of the interference patterns and/or processed data. The computer 20 includes a central processing unit (CPU) 21, a storage memory (RAM) 22, a user input/output (I/O) interface 23, and a system interface 24 which are all interconnected via a data bus 25. The computer 20 can programmed to issue a command that can be transmitted to the various parts of the OCT system 10 upon receiving a user input via the user interface 23. A key board, a mouse, and/or touch panel screen in the display 30 can be provided as part of the user interface 23.

The CPU 21 may be configured to read and perform computer-executable instructions stored in the storage memory 22. The computer-executable instructions may include those for the performance of the methods, measurements, and/or calculations described herein. For example, CPU 21 may receive signals from detector 17 and calculate, measure, or determine the intensity of the interference light and/or interference patterns.

The system interface 24 provides communication interfaces to input and output devices, which may include a keyboard, a display, a mouse, a printing device, a touch screen, a light pen, an optical storage device, a scanner, a microphone, a camera, a drive, communication cable and a network (either wired or wireless).

The optical-imaging device 19, located, for example, at the distal end of an OCT probe is usually comprised of a number of components designed to shape and direct light coming from and to the probe optical fiber as well as to maintain mechanical integrity of the probe. Examples of the optical probe too applicable to the optical-imaging device 19 include either of the probe 700 shown in FIG. 7, the probe 800 shown in FIG. 8, or the probe 900 shown in FIG. 9.

However, the optical-imaging device 19 and optical probe too described in the present disclosure are not limited to those applicable to an OCT system. The optical-imaging device 19 can be, for example, a catheter or an endoscope. In the case where the optical-imaging device 19 is an endoscope, the imaging system does not use an interferometer.

FIG. 10C illustrates an exemplary SEE system 50 for imaging bodily lumens with an SEE probe, as disclosed in any of the embodiments shown in FIGS. 1A to 6D. In this case, to acquire an intraluminal image with the SEE system 50, a microprocessor 94 is configured to control a light source 51 to output light of a broadband spectrum for illuminating a non-illustrated sample. The light is guided or otherwise transmitted by an optical system 92 to a fiber optic rotary joint (FORJ 93); thereafter, the light is delivered to a distal optics component 906 of a SEE probe too via an optical fiber (a light guiding component 102). The light guiding component 102 is arranged inside a drive cable 120 which extends from a proximal end to a distal end of the probe 100. The distal optics component 906 has a beam directing surface aligned with an optical axis of the light guiding component 102, and at least one surface of the distal optics component 906 is directly bonded to the distal end of the light guiding component 102 and/or the distal end of the drive cable 120. A light beam transmitted through the light guiding component 906 is directed and shaped by the beam directing surface of the distal optics component 906. As explained above, in an SEE probe, the light is dispersed to form a spectrally encoded line 56.

The light scattered back from dispersed line 56 formed in an object or sample (not shown) can be collected by detection fibers 59 and guided to a spectrometer 95. The detector/spectrometer 95 can be or include a line sensor, or it can include a simple light intensity detector such as photo-detector. By mechanically scanning the line, it is possible to acquire the two-dimensional image of the object. The detection fibers 59 may be arranged in between a non-rotatable inner sheath 125 and an outer sheath 58. The distal end of the probe 100 may have a transparent window 57. By mechanically scanning the SEE probe 100 in a rotating direction R using a mechanical scanning unit contained within the FORJ 93, it is possible to obtain a two-dimensional image of the object. The mechanical scan can be performed by, e.g., Galvo scanner or motor to rotate a drive cable 120 together with the light guiding component 102 and the distal optics 906 contained therein.

The SEE system 50 as described and shown with respect to FIG. 10C can diffract the broadband light along the axis of the probe 100, and facilitate forward viewing. The rotation of the probe is particularly useful for acquiring a two-dimensional front-view images of lumens, such as vessels. In some applications, it may be necessary that the probe 100 is to be rotated in an oscillating manner, e.g., +/−approximately 360 degrees back and forth, or +/−approximately 180 degrees back and forth, 90 degrees or 270 degrees of back and forth.

One function of the fiber junction (FORJ 93) is to make the SEE probe 100, including the illumination fiber 102 and distal optics 906, detachable. With this exemplary function, the probe 100 can be disposable and thus a sterile probe for human “in vivo” use can be provided every time an imaging operation is performed. In embodiments where the probe 100 is a disposable probe, the fiber 102, and/or the detection fibers 59 may be detachable. With this exemplary function, the probe 100 may be disposable in order to ensure that a sanitary probe is used in treating a subject which may be a human body. The microprocessor 94 shown in FIG. 10C has the same structure and substantially similar function as that of computer 20 shown in FIG. 10B.

While the present disclosure has been described with reference to exemplary embodiments, it is to be understood that the present disclosure is not limited to the disclosed exemplary embodiments. The scope of the following claims is to be accorded the broadest interpretation so as to encompass all such modifications and equivalent structures and functions.

In referring to the description, specific details are set forth in order to provide a thorough understanding of the examples disclosed. In other instances, well-known methods, procedures, components and circuits have not been described in detail as not to unnecessarily lengthen the present disclosure. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. An optical probe comprising:
a tubular shaft having an opening which extends from a proximal end to a distal end of the probe;
a light guiding component having at least a distal end thereof arranged at least partially inside the opening of the tubular shaft; and
a distal optics component,
wherein the distal optics component is formed directly molded at the distal end of the light guiding component and at the distal end of the tubular shaft, and
wherein the distal optics component has at least one beam directing surface and at least one surface directly bonded to the opening of the tubular shaft.

2. The optical probe according to claim 1,
wherein the distal optics component is directly bonded to the light guiding component and is at least partially molded inside the opening of the tubular shaft at the distal end thereof, and
wherein the tubular shaft is configured to rotate both the distal optics component and the light guiding component.

3. The optical probe according to claim 1,
wherein the distal optics component is arranged a predetermined distance from a distal end surface of the light guiding component and is at least partially molded inside the opening of the tubular shaft at the distal end thereof,
wherein the tubular shaft is configured to rotate the distal optics component while the light guiding component remains stationary.

4. The optical probe according to claim 1,
wherein the distal optics component comprises an optical-focusing component and a diffractive component, and
wherein the optical-focusing component and the diffractive component are integrally formed as one piece.

5. The optical probe according to claim 4,
wherein the light-guiding component and the optical-focusing component are aligned along a single optical axis; and
wherein a light passing through the light guiding component is incident on the beam directing surface, and thereafter the light is directed in a direction substantially non-parallel to the optical axis.

6. The optical probe according to claim 1,
wherein the distal optics component comprises an optical spacer having a first surface with optical power and a second surface with a diffractive element,
wherein the light guiding component is an optical fiber having a stripped portion, and
wherein the optical spacer is integrally formed as a single molded piece surrounding the stripped portion of the optical fiber.

7. The optical probe according to claim 6,
wherein the light-guiding component and the optical spacer are off-centered with respect to their respective optical axis; and
wherein a light passing through the light guiding component is incident on the first surface, is guided to the second surface, and thereafter the light is spectrally dispersed by the diffractive element in a forward direction such that at least part of the dispersed light propagates parallel to the optical axis.

8. The optical probe according to claim 1,
wherein the tubular shaft includes a drive cable,
wherein at least one surface of the distal optics component is directly bonded to the distal end of the light guiding component and/or to the distal end of the drive cable, and
wherein both the light guiding component and the distal optics component are adapted to rotate together with the drive cable.

9. The optical probe according to claim 1,
wherein the tubular shaft includes a drive cable and a cylindrical housing which is attached to the distal end of the drive cable, and
wherein the distal optics component is directly bonded inside the cylindrical housing.

10. The optical probe according to claim 9, wherein the cylindrical housing has a window configured to allow the light passing through the light guiding component and incident on the beam directing surface of the distal optics component to exit the optical probe in a direction angular to the optical axis.

11. The optical probe according to claim 10, wherein the distal optics component is integrally formed inside the cylindrical housing and abutting against the distal end of the light guiding component.

12. The optical probe according to claim 1, wherein the distal optics component further comprises a beam shaping surface.

13. The optical probe according to claim 1, wherein the distal optics component further comprises an atraumatic lead-in feature having a substantially rounded-off profile.

14. The optical probe according to claim 1, wherein the light-guiding component is comprised of an optical fiber and an optical spacer which is directly and coaxially affixed to the distal end of the optical fiber.

15. The optical probe according to claim 14,
wherein the tubular shaft includes a drive cable and a cylindrical housing, and
wherein the distal optics component is molded surrounding the distal end of the optical spacer and directly bonded inside the cylindrical housing.

16. The optical probe according to claim 1,
wherein the distal optics component is injection molded out of transparent thermoplastic material or compression molded out of glass.

17. An OCT (optical coherence tomography) imaging system, comprising:
an interferometer having a sample arm and a reference arm, the sample arm including the optical probe according to claim 1;
a fiber optic rotary junction (FORJ) configured to rotate and/or translate the optical probe inside a lumen,
a detector configured to detect an interference signal of a sample beam with a reference beam, the sample beam irradiating the lumen while the FORJ rotates and/or translates the optical probe, and
a processor configured to generate OCT images of the inside of the lumen.

18. An SEE (spectrally encoded endoscopy) imaging system, comprising:
a light source;
a detector;
the optical probe according to claim 1 in optical communication with the light source and the detector;
a fiber optic rotary junction (FORJ) configured to rotate and/or translate the optical probe inside a lumen; and
one or more processors configured to control and operate the light source, the detector, and the FORJ,
wherein the distal optics component includes a diffractive component in an optical path of the at least one beam directing surface,
wherein the optical probe is configured for guiding light from the light guiding component, through the distal optics component, and to the at least one beam directing surface, to the diffractive component, and thereafter forwarding a spectrally dispersed light line from the diffractive component towards an image plane,
wherein a distal optics component including the at least one beam directing surface and the diffractive component is arranged inside the drive cable such that at least one wavelength of the spectrally dispersed light line exits the probe substantially parallel to the longitudinal axis of the drive cable,
wherein the detector is configured to detect light reflected from the image plane while the FORJ rotates and/or translates the optical probe, and
wherein the one or more processors is configured to generate SEE images of the inside of the lumen.

19. A method of forming an optical probe, comprising:
inserting and positioning a distal end of a light guiding component inside a mold adapted to form a distal optics component having at least one beam directing surface;
injecting molten optically transparent material into the mold;
allowing time for the injected material to solidify; and
opening the mold and removing the distal end of the light guiding component with the distal optics component directly molded on the distal end of the light guiding component.

20. An imaging probe comprising:
a drive cable having a shape of a hollow shaft extending from a proximal end to a distal end of the probe;
a light guiding component arranged at least partially inside the hollow shaft of the drive cable; and
a distal optics component formed directly over the distal end of the light guiding component by overmolding,
wherein the distal optics component is directly molded over the distal end of the light guiding component and is at least partially molded at the distal end of the drive cable.

21. The optical probe according to claim 20, wherein the light-guiding component is comprised of an optical fiber and an optical spacer which is directly and coaxially affixed to the distal end of the optical fiber, and
wherein at least a portion of the distal optics component is molded at least partially over the optical spacer.

22. The optical probe according to claim 20, wherein the distal optics component includes, an optical spacer, a reflective surface, a focusing lens, and a lead-in end all formed as a single molded component which is molded at least partially inside the hollow shaft of the drive cable and directly over at least part of the light guiding component.

23. The optical probe according to claim 20,
further comprising a tubular mechanical housing bonded to the distal end of the hollow shaft of the drive cable,
wherein the distal optics component includes, an optical spacer, a reflective surface, a focusing lens, and a lead-in end all formed in a single part molded at least partially inside the mechanical housing and contacting directly the distal end of the light guiding component.

24. The optical probe according to claim 20,
wherein the distal optics component is injection molded out of transparent thermoplastic material or compression molded out of glass.

25. The optical probe according to claim 24,
wherein the distal optics component includes, an optical spacer which is molded at least partially inside the hollow shaft of the drive cable and molded directly over at least part of the light guiding component,
wherein, at the distal end thereof, the optical spacer includes a first surface and a second surface arranged at an angle with respect to each other,
wherein the first surface is or includes a reflective surface which reflects the light from the light guiding component towards the second surface by total-internal-reflection or by a mirror coating formed on the first surface,
wherein the second surface includes a diffractive component which is added by micro-stamping a suitable material onto the second surface to form a diffractive grating, and
wherein the diffractive grating disperses the light towards an imaging plane.
